(19) United States
(12) Patent Application Publication
(10) Pub. No.: US 2008/0207570 A1
(43) Pub. Date: Aug. 28, 2008

(54) Topically applicable oil-in-water emulsions and dermatological applications thereof

(75) Inventor: Sandrine Segura-Orsoni, Mandelieu (FR)

Correspondence Address: BUCHANAN, INGERSOLL & ROONEY PC, POST OFFICE BOX 1404, ALEXANDRIA, VA, 22313-1404, US

(73) Assignee: L'OREAL, PARIS (FR)

(21) Appl. No.: 12/068,670

(22) Filed: Feb. 8, 2008

Related U.S. Application Data
(63) Continuation of application No. PCT/FR2006/001930, filed on Aug. 9, 2006.

(30) Foreign Application Priority Data
Aug. 11, 2005 (FR) 0508524

(51) Int. Cl. A61K 31/59; A61K 47/06; A61K 31/573; A61P 17/06; A61K 31/58; A61K 31/56

(52) U.S. Cl. 514/168; 514/772; 514/181; 514/180; 514/174

(57) ABSTRACT

Novel topically applicable oil-in-water emulsions, with a high proportion of oily phase in the inner phase, combine the occlusive and emollient properties of an ointment without having the drawbacks of a greasy feel, while at the same time promoting the therapeutic properties of a biologically active agent contained therein; such emulsions are especially useful for the treatment of dermatological diseases, conditions or afflictions, notably psoriasis.

### CROSS-REFERENCE TO PRIORITY/PCT APPLICATIONS

This application claims priority under 35 U.S.C. §119 of FR 0508524, filed Aug. 11, 2005, and is a continuation of PCT/FR 2006/001930, filed Aug. 9, 2006 and designating the United States (published in the French language on Feb. 22, 2007 as WO 2007/020349 A1; the title and abstract were also published in English), each hereby expressly incorporated by reference in its entirety and each assigned to the assignee hereof.

### BACKGROUND OF THE INVENTION

1. Technical Field of the Invention

The present invention relates to novel topical compositions in the form of oil-in-water emulsions, with a high proportion of oily phase in the inner phase of the emulsion, combining the occlusive and emollient properties of an ointment without having the drawbacks of a greasy feel, while at the same time promoting the therapeutic properties of the biologically active agent present in said composition.

2. Description of Background and/or Related and/or Prior Art

For the targeted pathologies where the skin is dry and desquamated, the use of an ointment is widely preferred. In fact, the ointment is mainly composed of fatty substances which provide skin emollience and limit desquamation. However, this galenical form has the drawback, by virtue of its nature, of having a greasy and tacky feel, hence a discomfort in the patient's daily life (unpleasant sensations, greasy stains on clothing, etc.). The majority of these ointments are based on a high percentage of petroleum jelly.

EP-AI-0,069,423 describes, for example, compositions for topical application to the skin, in which the fatty phase is from 60% to 70% by weight.

Furthermore, emulsions comprising a continuous aqueous phase are known which offer the advantage that they do not have a greasy feel, they are very easy to spread, etc. However, the emollient capacity of such emulsions is very often insufficient, or even unsatisfactory.

Need therefore exists for compositions having both the advantages of ointments (occlusivity, emollience) and of emulsions in a continuous aqueous phase (non-greasy feel, cosmetically acceptable, ease of spreading, no residues), while at the same time avoiding the drawbacks associated with these two types of formulation.

In addition, in terms of effectiveness, such compositions should promote the therapeutic properties of the biologically active agent, in order to guarantee its effectiveness in this new galenical form.

Advantageously, when they comprise at least one therapeutic agent of the corticosteroid class, these compositions should have a bioequivalence in terms of vasoconstriction with the major products commercially available, in particular for corticosteroid-based topical compositions.

### SUMMARY OF THE INVENTION

Novel oil-in-water emulsions containing a very high proportion of fatty phase have now been developed, corresponding to the proportions generally contained in water-in-oil emulsions.

Such novel emulsions, also deemed lipocreams, comprise a nonionic emulsifying system of oil-in-water type, combined with one or more propenetrating agents.

Said emulsions comprising a nonionic emulsifying system of oil-in-water type make it possible, despite the very substantial inner fatty phase, not to change the direction of the emulsion.

Consequently, the emulsions comprising a continuous aqueous phase according to the invention presents the advantage of having a non-greasy feel while at the same time offering a satisfactory emollient capacity provided by the high percentage of fatty phase.

Surprisingly, these novel formulations have a therapeutic activity equivalent to that expected in the case of ointments having a high concentration of petroleum jelly.

Thus, the present invention features, firstly, oil-in-water emulsions containing at least one therapeutic agent for topical application and also comprising:

a) from 25% to 60% by weight of a fatty phase;

b) from 1% to 15% by weight of a nonionic emulsifying system;

c) from 1% to 30% by weight of at least one propenetrating agent; and

d) from 5% to 50% by weight of water.

Advantageously, the emulsions according to the present invention comprise an amount of fatty phase of from 35% to 50% by weight.

### DETAILED DESCRIPTION OF BEST MODE AND SPECIFIC/PREFERRED EMBODIMENTS OF THE INVENTION

For the purpose of the present invention, the term “fatty phase” means one or more compounds selected from among emollients, waxes and fatty alcohols, alone or as mixtures of two or more thereof.

The emollients will advantageously be the emollients commonly employed for the formulations in accordance with the present invention and well known to one skilled in this art. By way of non-limiting examples, representative are plant oils, such as cottonseed oil or sweet almond oil; esters, such as isopropyl palmitate and isopropyl myristate; mineral oils, such as light mineral oils, volatile or non-volatile silicone oils, for instance dimethicone and cyclomethicone; and petroleum jelly.

The waxes that may also be used as constituents of the fatty phase of the emulsions according to the present invention are the waxes commonly employed by one skilled in this art in this field, among which are, purely by way of illustration, beeswax, carnauba wax, ozokerite, paraffin and dimethiconol behenate.

Similarly, the fatty phase may contain one or more fatty alcohols known to one skilled in this art and, in this respect, exemplary are cetyl alcohol and stearyl alcohol.

The fatty phase will preferably contain one or more of the following ingredients: isopropyl palmitate, a mineral oil, petroleum jelly, dimethicone.

According to the present invention, the fatty phase is emulsified by means of a nonionic emulsifying system.

Surfactants are described as amphiphilic molecules because of their double hydrophilic and lipophilic polarity. The structure of these molecules makes it possible to characterize them: the hydrophilic-lipophilic balance (HLB) is the ratio of the hydrophilic part to the lipophilic part. A high HLB indicates that the hydrophilic fraction is predominant and, conversely, a low HLB indicates that the lipophilic part is predominant. For example, HLB values greater than approximately 10 correspond to hydrophilic surfactants.

Surfactants can be categorized, according to their structure, under the generic terms “ionic” (anionic, cationic, amphoteric) or “nonionic”. Nonionic surfactants are surfactants which do not dissociate to ions in water and are therefore insensitive to variations in pH.

Nonionic surfactants are particularly suitable for the preparation of the oil-in water emulsions of the present invention. Thus, the emulsifying system, a component of the emulsions of the invention, comprises at least one nonionic surfactant with a predominant hydrophilic fraction, i.e., having a high HLB, greater than approximately 10.

By way of examples of nonionic surfactants having a high HLB, representative are sorbitan esters such as POE(20) sorbitan monooleate, marketed under the trademark “Tween 80” (HLB=15); POE(20) sorbitan monostearate marketed under the trademark “Tween 60” (HLB=14.9); fatty alcohol ethers such as POE(21) stearyl ether (HLB=15.5), or ceteareth 20 marketed under the trademark “Eumulgin B2” by Cognis (HLB of 15.5).

Preferably, said high-HLB nonionic surfactants have an HLB of from 10 to 18.

As examples of nonionic surfactants with a low HLB, representative are sorbitan esters, such as sorbitan monostearate (marketed under the trademark Span 60 by Unichema), glycerol esters (marketed under the trademark Cutina GMSVPH by Cognis) such as glyceryl monostearate (Cutina GMS from Cognis), and low-HLB sucrose esters such as sucrose distearate.

Preferably, said nonionic surfactants with a low HLB have an HLB of less than 10.

The nonionic surfactants can be used alone or as a mixture of two or more of same so as to form the emulsifying system constituting the emulsion of the invention.

One or more “high-HLB nonionic surfactant”/“low-HLB nonionic surfactant” couples will preferably be employed as emulsifying system; it may in particular be a nonionic emulsifying system comprising at least surfactant having an HLB of greater than approximately 10 and at least one nonionic surfactant having an HLB of less than approximately 10.

According to a particularly preferred embodiment of the present invention, the emulsifying system is composed of the glyceryl monostearate/ceteareth 20 surfactant couple.

The use of the abovementioned surfactant couples offers the advantage of rigidifying the interfacial film and, consequently, of substantially increasing the stability of the emulsion.

The ratio of each of the two surfactants constituting the abovementioned couple is determined most commonly by calculating the required HLB of the fatty phase employed.

Determination of the optimal emulsification of a fatty substance or of a mixture of fatty substances involves the prior determination of its required HLB. In fact, each fatty substance has a defined required HLB. It is not possible therefore to utilize the same emulsifier in all cases. The HLB required by the fatty phase corresponds to the value of the HLB of the emulsifier or of the emulsifier couple which will provide, with the fatty substances considered, the most stable emulsion.

In addition to the emulsifying system which has just been described, the emulsions of the present invention comprise one or more propenetrating agents in preferred concentrations ranging from 1% to 30%, preferably from 5% to 25%, and more preferably ranging from 15% to 20% by weight relative to the total weight of the composition.

The propenetrating agents must generally not solubilize the therapeutic agents at the percentage used, not cause exothermic reactions that are harmful to the various constituents, help with good dispersing of said therapeutic agents and have anti-foam properties. Among the propenetrating agents, preferably employed, without this list being limiting, are the compounds selected from those known to one skilled in this art, such as propylene glycol, dipropylene glycol, Transcutol® (ethoxydiglycol), propylene glycol dipelargonate, lauroglycol, urea, acetone or oleic acid.

The propenetrating agents can of course be formulated as mixtures of two or more of same.

The propenetrating agent is preferably selected from propylene glycol, dipropylene glycol and propylene glycol dipelargonate. More preferably, the propenetrating agent is propylene glycol.

It should be understood that the nature of the propenetrating agent will depend on the nature of the therapeutic agent included in the emulsion of the invention.

The aqueous phase of the emulsions according to the invention comprises water, which may or may not be purified and which may or may not be demineralized, and in particular a floral water such as cornflour water, or a natural mineral water or spring water, for example selected from eau de Vittel, waters from the Vichy basin, eau d'Uriage, eau de la Roche-Posay, eau de la Bourboule, eau d'Enghien-les-Bains, eau de Saint Gervais-les-Bains, eau de Néris-les-Bains, eau d'Allevard-les-Bains, eau de Digne, eau de Maizières, eau de Neyrac-les-Bains, eau de Lons-le-Saunier, les Eaux Bonnes, eau de Rochefort, eau de Saint Christau, eau des Fumades, eau de Tercis-les-Bains, eau d'Avene or eau d'Aix les Bains.

Thus, in addition to the fatty phase, the emulsifying system, the propenetrating agent and the therapeutic agent, the emulsions according to the present invention comprise a substantial amount of water, from 5% to 50%, advantageously approximately 30% by weight of the emulsion.

According to a preferred embodiment, the oil-in-water emulsions of the present invention are most particularly suitable for the treatment and/or prevention of skin diseases. The therapeutic agent included in said emulsion will consequently preferably be a compound that is therapeutically active in the treatment and/or prevention of diseases of this type.

The emulsions of the present invention have been found to be particularly effective for the topical application to the corticosteroid class that are already known and commercially available.

Examples of the therapeutic agents of the corticosteroid class are clobetasol 17-propionate, desonide, betamethasone, betamethasone acetate, betamethasone valerate, betamethasone dipropionate, betamethasone dipropionate monohydrate, diflucortolone valerate, fluticasone valerate, hydrocortisone 17-butyrate, mometasone furoate, halobetasol propionate, desoxymetasone, clobetasone butyrate, hydrocortisone, cortisone, prednisolone, miconazole, prednisone, triamcinolone acetonide, methylprednisolone, fluometholone, fluocinolone acetonide, dexamethasone, dexamethasone dipropionate, aclasone, dexamethasone 17,21-dipropionate, diflorasone diacetate, or mixtures thereof.

The therapeutic agents of the corticosteroid class will preferably be selected from among clobetasol 17-propionate, desonide and betamethasone valerate, clobetasol 17-propionate being the preferred therapeutic agent.

Of course, the present invention is not limited to these corticosteroids only, and many other therapeutic agents and other therapeutic applications can be envisaged in the context of topical applications by means of oil-in-water emulsions.

Thus, the emulsions of the present invention can also be used for formulations for topical applications to the skin of vitamin D and of analogues thereof.

Examples of vitamin D analogues are ergocalciferol, alfacalcidol, calcifediol, calcipotriol, calcitriol, cholecalciferol, tacalcitol, 6-(3-hydroxy-5,5,8,8-tetramethyl-5,6,7,8-tetrahydronaphthalen-2-ylselanyl)nicotinic acid, 4-[6-ethyl-4′-(1-ethyl-1-hydroxypropyl)-2′-propylbiphenyl-3-yloxymethyl]-2-hydroxymethylphenyl}methanol, or mixtures thereof.

The emulsions of the present invention may also be used for formulations for topical applications to the skin of retinoids.

The term “retinoid” means any compound which binds to RAR and/or RXR receptors.

Preferably, the retinoid is a compound selected from the family of benzonaphthalene retinoids as described in EP-0,199,636. In particular, preference will be given to adapalene (6-[3-(1-adamantyl)-4-methoxyphenyl]-2-naphthanoic acid), and also precursors and/or derivatives thereof. Tretinoin and isotretinoin may also be used.

The term “retinoid precursors” means the immediate or substrate biological precursors of retinoids, and also the chemical precursors of retinoids.

The term “retinoid derivatives” means both metabolic derivatives of retinoids and chemical derivatives of retinoids.

Other retinoids can be selected from among those described in the following patents or patent applications: U.S. Pat. Nos. 4,666,941, 4,581,380, EP-0,210,929, EP-0,232,199, EP-0,260,162, EP-0,292,348, EP-0,325,540, EP-0,359,621, EP-0,409,728, EP-0,409,740, EP-0,552,282, EP-0,584,191, EP-0,514,264, EP-0,514,269, EP-0,661,260, EP-0,661,258, EP-0,658,553, EP-0,679,628, EP-0,679,631, EP-0,679,630, EP-0,708,100, EP-0,709,382, EP-0,722,928, EP-0,728,739, EP-0,732,328, EP-0,740,937, EP-0,776,885, EP-0,776,881, EP-0,823,903, EP-0,832,057, EP-0,832,081, EP-0,816,352, EP-0,826,657, EP-0,874,626, EP-0,934,295, EP-0,915,823, EP-0,882,033, EP-0,850,909, EP-0,879,814, EP-0,952,974, EP-0,905,118, EP-0,947,496, W098/56783, W099/10322, W099/50239 and W099/65872.

According to another embodiment, the oil-in-water emulsions of the present invention may be prepared with a combination of various therapeutic agents selected from the various categories described above.

One of the main objectives of the invention is to provide a system for topical application which has the advantages of a fatty cream, as regards the properties of penetration through the epidermis, and also the advantages of an aqueous emulsion, from the point of view of comfort and ease with which it is used, and also from the point of view of the stability of said formulation.

The emulsions described herein and which are the subject of the present invention may also comprise any additive normally employed in the cosmetics or pharmaceutical field, such as sequestering agents, antioxidants, sunscreens, preservatives, fillers, electrolytes, humectants, pH buffers, dyes, common inorganic or organic acids or bases, fragrances, essential oils, active cosmetic agents, moisturizers, vitamins, sphingolipids, self-tanning compounds, such as DHA, gelling agents, and agents for soothing and protecting the skin such as allantoin.

Of course, one skilled in this art will take care to select this or these possible additional compound(s), and/or the amount thereof, in such a way that the advantageous properties of the emulsions according to the invention are not, or not substantially, impaired.

Purely by way of illustration, as examples of sequestering agents, mention may be made of ethylenediaminetetraacetic acid (EDTA) and derivatives or salts thereof, dihydroxyethylglycine, citric acid and tartaric acid, or mixtures thereof.

As examples of preservatives, mention is made of benzalkonium chloride, phenoxyethanol, benzyl alcohol, diazolidinylurea and parabens, or mixtures thereof.

Among the antioxidants, mention is, by way of non-limiting examples, made of ascorbic acid and salts thereof, tocopherols and the sulfite salts such as sodium metabisulfite or sodium sulfite, butylhydroxyanisole, butylhydroxytoluene and propyl gallate.

Preferably, the antioxidant is selected from DL-alpha-tocopherol, butylhydroxyanisole, butylhydroxy-toluene and propyl gallate.

As examples of humectants, mention is made of glycerol and sorbitol, and as pH buffers, mention is made of citric acid and sodium citrate.

The emulsions according to the invention may also contain, as additive, one or more compounds under the category of gelling agents, i.e., capable of conferring on the emulsion a sufficient viscosity to maintain the various constituents of said emulsion in suspension.

These gelling agents may advantageously be selected from commonly used gelling agents, and in particular selected from, by way of non-limiting examples, carbomer, hydroxyethylcellulose, methylcellulose, guar gum, xanthan gum, hectorite, pectin, magnesium aluminum silicate, gelling agents of the polyacrylamide family, such as the sodium acryloyldimethyltaurate/isohexadecane/polysorbate 80 copolymer blend marketed under the trademark Simulgel 600® by Seppic, the polyacrylamide/C13-14 isoparaffin/laureth-7 mixture such as, for example, that marketed under the trademark Sepigel 3050 by Seppic, the family of acrylic polymers coupled to hydrophobic chains, such as the PEG150/decyl/SMDI copolymer marketed under the trademark Aculyn 44® (polycondensate comprising at least, as elements, one polyethylene glycol comprising 150 or 180 mol of ethylene oxide, decyl alcohol and methylenebis(4-cyclohexylisocyanate) (SMDI), at 35% by weight in a mixture of propylene glycol (39%) and water (26%)), the family of modified starches such as the modified potato starch marketed under the trademark Structure Solanace, or else mixtures of two or more of these.

The preferred gelling agents are xanthan gum, magnesium aluminum silicate (Veegum K from Vanderbilt), magnesium aluminum silicate/aluminum/titanium dioxide (Veegum ULTRA from Vanderbilt), or mixtures thereof.

According to a preferred embodiment, the oil-in-water emulsion of the present invention comprises:

a) from 35% to 45% by weight of a fatty phase;

b) from 5% to 15% by weight of a nonionic emulsifying system;

c) from 15% to 20% by weight of at least one propenetrating agent;

d) from 0.01% to 0.5% by weight of at least one therapeutic agent;

e) from 5% to 50% by weight of water; and

f) additives, preferably from 0.2% to 25% by weight.

According to another embodiment, the present invention features a method for preparing the oil-in-water emulsions as defined above, which comprises the steps of:

a) dissolution and/or dispersion in water, with stirring, of at least one propenetrating agent and of the optional additive(s), at a temperature of from 60° C. and 95° C., until a homogeneous aqueous phase is obtained;

b) incorporation, with stirring, of the aqueous phase into the fatty phase, in which the emulsifier couple has been solubilized, preheated to a temperature of from 60° C. and 95° C.; and

c) incorporation, with stirring, and at a temperature of less than 40° C., of the therapeutic agent, optionally presolubilized in an appropriate solvent.

The term “appropriate solvent” means any solvent capable of solubilizing the therapeutic agent(s) and/or of dissolving the therapeutic agent(s), when the latter is (are) in solid form, so as to allow them to be perfectly incorporated into the fatty phase/aqueous phase emulsion.

According to yet another embodiment, the present invention features a physiologically acceptable galenical formulation compatible with topical application to the skin, the integuments or the mucous membranes, comprising at least one oil-in-water emulsion as defined above. According to one embodiment, the galenical form is an oil-in-water emulsion per se.

The invention also features the use of the emulsion described above or of the abovementioned formulation for the application to the skin, the integuments or the mucous membranes of at least one therapeutic agent belonging to the corticosteroid class, in particular with a view to the prevention and/or treatment of dermatological diseases, conditions or afflictions (whether regime or regimen).

Said therapeutic agents are particularly suitable in the following treatment fields:

1) for treating dermatological conditions linked to a keratinization disorder related to differentiation and proliferation, in particular for treating common acne, comedone-type acne, polymorphic acne, acne rosacea, nodulocystic acne, acne conglobata, senile acne, secondary acne such as solar acne, acne medicamentosa or occupational acne,

2) for treating other types of keratinization disorders, in particular ichthyosis, ichthyosiform states, Darrier's disease, palmoplantar keratoderma, leucoplasia and leucoplasiform states, cutaneous or mucosal (buccal) lichen,

3) for treating other dermatological conditions with an inflammatory immunoallergic component, with or without cell proliferation disorder, and in particular all the forms of psoriasis, whether cutaneous, mucosal or ungual, and even psoriatic rheumatism, or cutaneous atopy, such as eczema or respiratory atopy or gingival hypertrophy,

4) for treating any dermal or epidermal proliferations whether benign or malignant, whether of viral or non-viral origin, such as verruca vulgaris, verruca plana and epidermodysplasia verruciformis, oral or florid papillomatoses, T lymphoma, and proliferations which may be induced by ultraviolet radiation, in particular in the case of baso- and spinocellular epitheliomas, and any precancerous skin lesion such as keratoacanthomas,

5) for treating other dermatological disorders such as immune dermatoses, such as lupus erythematosus, bullous immune diseases and collagen diseases, such as scleroderma,

6) in the treatment of dermatological or general conditions with an immunological component,

7) in the treatment of skin disorders due to exposure to UV radiation and for repairing or combating skin aging, whether photoinduced or chronological, or for reducing actinic keratoses and pigmentations, or any pathologies associated with chronological or actinic aging, such as xerosis,

8) for combating sebaceous function disorders such as acne hyperseborrhea, simple seborrhea, or seborrheic dermatitis,

9) for preventing or treating cicatrization disorders, or for preventing or repairing stretch marks,

10) in the treatment of pigmentation disorders, such as hyperpigmentation, melasma, hypopigmentation or vitiligo,

11) in the prevention or treatment of alopecia of various origins, in particular alopecia due to chemotherapy or to radiation.

Among the dermatological diseases, the oil-in-water emulsions of the present invention have been found to be entirely suitable for the prevention and treatment of all the forms of psoriasis, whether cutaneous, mucosal or ungual.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES

### Example 1

### Protocol

Formulations 1, 2, 3 and 4 presented in the following Examples 2, 3, 4 and 5 were obtained according to the protocol described below.

Preparation of an Oil-in-Water Emulsion:

The fatty phase is weighed into a recipient beaker, which is then placed in a waterbath at 78° C.

The aqueous phase is weighed into a beaker, and citric acid is then added. Sodium citrate is then dispersed with vigorous stirring.

The gelling agents are then added, followed, after homogenization, by propylene glycol, with the mixture being heated to 78° C. with Rayneri mixing.

The hot fatty phase is then stirred in a Rayneri mixer. The aqueous phase is poured into the fatty phase with stirring (1,000 rpm) and heating at 78° C. for 10 min. The mixture is left to stir (1,000 rpm) without heating.

During this time, the therapeutic agent is prepared by adding it, with mechanical stirring, in a beaker, to propylene glycol, until complete dissolution is obtained.

This active phase is then incorporated into the emulsion, below 40° C.

### Example 2

### Formulation 1

| Composition | % by weight |
|---|---|
| Ceteareth 20 | 3 |
| Glyceryl monostearate | 5 |
| Cetyl alcohol | 0.5 |
| Mineral oil | 10.00 |
| Sweet almond oil | 3.00 |
| Petroleum jelly | 7.00 |
| Isopropyl myristate | 14.00 |
| Benzyl alcohol | 0.50 |
| Purified water | qs 100.00 |
| Hydroxyethylcellulose | 0.70 |
| Xanthan gum | 0.20 |
| Ethoxydiglycol | 8.00 |
| Desonide | 0.05 |

### Example 3

### Formulation 2

| Composition | % by weight |
|---|---|
| Ceteareth 20 | 2.50 |
| Glyceryl monostearate | 6.00 |
| Light mineral oil | 10.00 |
| Caprylic/capric triglycerides | 10.00 |
| Isopropyl palmitate | 15.00 |
| Dimethicone | 1.00 |
| Stearoxytrimethylsilane and stearyl alcohol | 0.80 |
| Propylparaben | 0.20 |
| Purified water | qs 100.00 |
| Citric acid | 0.140 |
| Sodium citrate | 0.294 |
| Methylparaben | 0.20 |
| Magnesium aluminum silicate/aluminum/TiO₂ | 0.80 |
| Xanthan gum | 0.22 |
| Propylene glycol | 17.00 |
| Clobetasol 17-propionate | 0.05 |

### Example 4

### Formulation 3

| Composition | % by weight |
|---|---|
| Ceteareth 20 | 3.75 |
| Glyceryl monostearate | 6.25 |
| Cetostearyl alcohol | 1.00 |
| Mineral oil | 15.00 |
| Petroleum jelly | 11.00 |
| Isopropyl palmitate | 13.00 |
| Dimethicone | 1.00 |
| Propylparaben | 0.20 |
| Purified water | 30.096 |
| Citric acid | 0.140 |
| Sodium citrate | 0.294 |
| Methylparaben | 0.20 |
| Magnesium aluminum silicate/aluminum/TiO₂ | 0.80 |
| Xanthan gum | 0.22 |
| Propylene glycol | 17.00 |
| Clobetasol 17-propionate | 0.05 |

Physical Stability of Formulation 3:

At ambient temperature (Ta), macroscopic observation makes it possible to guarantee the physical integrity of the products and microscopic observation makes it possible to verify that there is no recrystallization of the solubilized active agent and no significant change in the size of the globules of the emulsion.

At 4° C., microscopic observation verifies that there is no recrystallization of the solubilized active agent.

At 45° C., macroscopic observation verifies the integrity of the finished product.

The results are reported in the following table:

| Storage | Storage | Haake flow | | |
|---|---|---|---|---|
| conditions | period | threshold | pH | Appearance |
| Ta | t = 0 | Tau 0 = 49 Pa · s⁻¹ | 5.28 | Smooth white |
| | | | | cream |
| | t = 1 month | Tau 0 = 48 Pa · s⁻¹ | 5.19 | idem |
| | t = 2 months | Tau 0 = 46 Pa · s⁻¹ | 5.28 | idem |
| | t = 4 months | Tau 0 = 45 Pa · s⁻¹ | 5.23 | idem |
| | t = 6 months | Tau 0 = 40 Pa · s | 5.23 | idem |
| T45° C. | t = 1 month | Not applicable | NA | complies |
| | t = 2 months | NA | NA | complies |
| | t = 4 months | NA | NA | complies |
| T4° C. | t = 1 month | NA | NA | complies |
| | t = 2 months | NA | NA | complies |

Rheological Data:

A Haake® VT500 rheometer with an SVDIN measuring sensor is used. The rheograms were produced at 25° C. by varying the shear rate over time and measuring the stress.

The term “flow threshold” (tau 0) means the force necessary (minimum shear stress) to overcome the Van der Waals type cohesion forces and bring about flow. The flow threshold is related to the value found at the shear rate of 4 s⁻¹.

Chemical Stability:

The chemical stability is an assay, by HPLC, of the active agent and of the preservatives at ambient temperature (Ta) and at 45° C.

The data are reported in the following tables:

Clobetasol Propionate:

| | t = 0 | t = 1 month | t = 2 months | t = 3 months |
|---|---|---|---|---|
| Ta | 99.5% | 99.9% | 99.4% | 100.0% |
| T45° C. | | 100.2% | 99.2% | 98.9% |

Methylparaben:

| | t = 0 | t = 1 month | t = 2 months | t = 3 months |
|---|---|---|---|---|
| Ta | 97.8% | 98.6% | 98.2% | 97.4% |
| T45° C. | | 97.9% | 98.6% | 97.8% |

Propylparaben:

| | t = 0 | t = 1 month | t = 2 months | t = 3 months |
|---|---|---|---|---|
| Ta | 98.2% | 99.3% | 98.9% | 100.4% |
| T45° C. | | 99.1% | 99.9% | 101.7% |

### Example 5

### Formulation 4

| Composition | % by weight |
|---|---|
| Ceteareth 20 | 3.75 |
| Glyceryl monostearate | 6.25 |
| Cetostearyl alcohol | 1.00 |
| Mineral oil | 15.00 |
| Petroleum jelly | 11.00 |
| Isopropyl palmitate | 13.00 |
| Dimethicone | 1.00 |
| Propylparaben | 0.05 |
| Purified water | 30.346 |
| Citric acid | 0.140 |
| Sodium citrate | 0.294 |
| Methylparaben | 0.10 |
| Magnesium aluminum silicate | 0.80 |
| Xanthan gum | 0.22 |
| Propylene glycol | 17.00 |
| Clobetasol 17-propionate | 0.05 |

Physical and chemical stability of the formulation relating to Example 5 according to the established specifications:

| Storage | Storage | Clobetasol 17- | | | Brookfield | | |
|---|---|---|---|---|---|---|---|
| conditions | period | propionate | Methylparaben | Propylparaben | viscosity | pH | Appearance |
| Stability | | 0.045% to | 0.09% to | 0.045% to | Results | 4.8 to 5.8 | White to pale |
| characteristics | | 0.055% | 0.11% | 0.055% | mPa · s | | yellowy |
| | | by weight | by weight | by weight | | | cream |
| 25° C. | t = 0 | 0.053 | 0.10 | 0.051 | 11871 | 5.5 | Complies |
| 60% | t = 1 | 0.054 | 0.10 | 0.052 | 11754 | 5.5 | Complies |
| relative | month | | | | | | |
| humidity | t = 3 | 0.052 | 0.10 | 0.051 | 12423 | 5.6 | Complies |
| | months | | | | | | |
| | t = 6 | 0.054 | 0.10 | 0.051 | 11986 | 5.6 | Complies |
| | months | | | | | | |
| | t = 10 | 0.052 | 0.10 | 0.051 | 13531 | 5.6 | Complies |
| | months | | | | | | |
| 40° C. | t = 1 | 0.055 | 0.10 | 0.052 | 11653 | 5.5 | Complies |
| 75% | month | | | | | | |
| relative | t = 3 | 0.053 | 0.10 | 0.051 | 13287 | 5.7 | Complies |
| humidity | months | | | | | | |
| | t = 6 | 0.054 | 0.10 | 0.051 | 16705 | 5.8 | Complies |
| | months | | | | | | |

### Example 6

### Formulation Prepared with a Vitamin D Analogue as Therapeutic Agent

| Composition | % by weight |
|---|---|
| Ceteareth 20 | 3.75 |
| Glyceryl monostearate | 6.25 |
| Caprylic/capric triglycerides | 9.00 |
| White soft paraffin | 3.00 |
| Isopropyl palmitate | 14.00 |
| Dimethicone | 2.00 |
| Propylparaben | 0.05 |
| dl-alpha-tocopherol | 0.07 |
| C₁₂-C₁₅ alkyl benzoate | 12.00 |
| 6-(3-Hydroxy-5,5,8,8-tetra-methyl- | 0.3 |
| 5,6,7,8-tetrahydronaphthalen-2- | |
| ylselany1)-nicotinic acid | |
| Purified water | qs 100.00 |
| Methylparaben | 0.2 |
| Magnesium aluminum silicate | 0.80 |
| Xanthan gum | 0.22 |
| Propylene glycol | 17.00 |
| BHA | 0.01 |

This formulation is in accordance with the subject of the present invention: no recrystallization of the therapeutic agent was observed after 3 months at 4° C.

### Example 7

### Evaluation of the Vasoconstrictor Effect of the Emulsions

The evaluation tests are carried out on formulations 2, 3 and 4 of Examples 3, 4 and 5 and, by way of comparison, with the commercial products Temovate Cream® and Temovate Emollient Cream®.

The study was carried out on 7 normal male or female individuals, 18 to 70 years old.

Methodology:

The study is carried out over 2 days. The three formulations and the two reference products were applied to each volunteer.

An area receiving no product served as a control area. On each volunteer and on each area studied (3 areas per forearm), the following evaluations were carried out:

basal clinical marks for vasoconstriction and basal colorimetry measurements;

application of the product to each area treated (area of 1.5 cm², 10 μ1 of product/area) and then removal of the excess 4 hours later;

clinical marks for vasoconstriction and colorimetry measurements 4 hours and 20 hours after removal of the excess, i.e., at DOT8 and D1T24.

Evaluation Criteria:

Main criterion: Clinical evaluation of vasoconstriction according to a scale of 0 to 4.

Secondary criterion: Biophysical measurements of colorimetry: a* and L*.

Statistical Analyses:

The variables obtained were subjected to an analysis of variance and each formulation was compared with the non-treated site and with the reference product at each measurement time.

Results:

All the products tested exhibit maximum whitening 4 hours after removal of the excess.

The two reference products and the three formulations tested had different whitening kinetics.

It is observed that Temovate Cream® induces the most substantial whitening. Statistically, the AUC for Temovate Cream® is significantly different from the AUC for the non-treated area and for formulation 2 of Example 3 for the parameter a*. For the parameter L*, the AUC for Temovate Cream® is significantly different from the AUC for all the other test areas, except for that treated with Temovate Emollient Cream®.

It is also observed that the following 3 products: Temovate Emollient Cream®, formulation 3 of Example 4 and formulation 2 of Example 3 are quite similar to one another. The AUCs for these 3 products are all significantly different from the AUC for the non-treated area for the parameter a*. For the parameter L*, only the AUC for the formulation of Example 3 is not different from the non-treated area.

The AUC for formulation 2 of Example 3 appears to be the smallest both for the parameter a* and for the parameter L*. Statistically, the AUC for the latter appears to be significantly different from the non-treated area for the parameter a*, but not for the parameter L*.

In terms of statistics, the Student's t test was carried out, with a significance threshold of less than 0.1.

Conclusion:

The formulations of Examples 3, 4 and 5 therefore exhibit vasoconstrictor effects equivalent to ointment-type products on the market (Temovate Cream®).

### Example 8

### Evaluation of the Irritant Effect of the Emulsions

Principle of the Test:

This test makes it possible to evaluate the irritant potential of formulation 2 and of formulation 3 in various irritation tests in normal volunteers.

The study was carried out on 12 normal individuals, 18 to 55 years old.

The treatment was carried out on various areas, as follows:
“elbow crease” areas: 1 application daily for 14 days.
“stripping” areas: 1 application daily for 7 days.
“patch” areas: 1 single application for 20 hours.

Methodology:

The study took place over 15 days for each volunteer.

The volunteers came to the test center on all the days of the study, except on D4, D8, D16, D11 and D12, where the applications took place at home.

On each volunteer, 3 areas of 4×4 cm were delimited on the inner face of the forearms, including one area in the elbow crease. The two formulations were applied to the left or right side according to a randomization procedure generated before the study.

Z1-Z4 (“elbow crease” areas): daily application of the products for 14 days at a rate of 2 mg/cm².

Z2-Z5 (“stripping” areas): 20 strippings were carried out using D-squame on DO, followed by daily application of the products for 7 days at a rate of 2 mg/cm².

Z3-Z6 (“patch” areas): single application of the products in an aluminum cupule (Finn Chambers®) 12 mm in diameter for a minimum of 20 h.

Results:

Tests carried out as indicated above show that the formulations of Examples 3 and 4 according to the present invention do not exhibit any irritation phenomenon.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A topically applicable oil-in-water emulsion containing at least one biologically active agent and also comprising:
a) a fatty phase, the amount thereof ranging from about 35% to 50% by weight;
b) from about 1% to 15% by weight of a nonionic emulsifying system;
c) from about 1% to 30% by weight of at least one propenetrating agent; and
d) from about 5% to 50% by weight of water.

2. The oil-in-water emulsion as defined by claim 1, in which the fatty phase consists essentially of emollients, waxes and fatty alcohols.

3. The oil-in-water emulsion as defined by claim 1, in which the nonionic emulsifying system comprises at least one nonionic surfactant having an HLB of greater than approximately 10 and at least one nonionic surfactant having an HLB of less than approximately 10.

4. The oil-in-water emulsion as defined by claim 1, in which the emulsifying system comprises the glyceryl monostearate/ceteareth surfactant couple.

5. The oil-in-water emulsion as defined by claim 1, in which the propenetrating agent is selected from among propylene glycol, dipropylene glycol, propylene glycol dipelargonate, lauroglycol, ethoxydiglycol, urea, acetone, oleic acid, and mixtures thereof.

6. The oil-in-water emulsion as defined by claim 1, in which the biologically active agent is for the treatment and/or prevention of skin diseases.

7. The oil-in-water-emulsion as defined by claim 1, in which the biologically active agent comprises a corticosteroid.

8. The oil-in-water emulsion as defined by claim 7, in which the biologically active agent comprises clobetasol 17-propionate, desonide or betamethasone valerate.

9. The oil-in-water emulsion as defined by claim 1, in which the biologically active agent comprises a retinoid.

10. The oil-in-water emulsion as defined by claim 1, in which the biologically active agent comprises a vitamin D analogue.

11. The oil-in-water emulsion as defined by claim 1, in which the amount of water is from approximately 30% by weight of the emulsion.

12. The oil-in-water emulsion as defined by claim 1, further comprising one or more additives.

13. The oil-in-water emulsion as defined by claim 12, further comprising at least one additive selected from the group consisting of sequestering agents, antioxidants, sunscreens, preservatives, fillers, electrolytes, humectants, pH buffers, dyes, inorganic or organic acids or bases, fragrances, essential oils, active cosmetic agents, moisturizers, vitamins, sphingolipids, self-tanning compounds, gelling agents, and agents for soothing and protecting the skin.

14. The oil-in-water emulsion as defined by claim 1, comprising:
a) from about 35% to 45% by weight of a fatty phase;
b) from about 5% to 15% by weight of a nonionic emulsifying system;
c) from about 15% to 20% by weight of at least one propenetrating agent;
d) at least one additives;
e) from about 0.01% to 0.5% by weight of at least one biologically active agent; and
f) from about 5% to 50% by weight of water.

15. A method for preparing an oil-in-water emulsion as defined by claim 1, comprising the steps of:
a. dissolution and/or dispersion in water, with stirring, of at least one propenetrating agent and of, optionally, at least one additive, at a temperature of from 60° C. and 95° C., until a homogeneous aqueous phase is obtained;
b. incorporation, with stirring, of the aqueous phase into the fatty phase, in which the emulsifier has been solubilized, preheated to a temperature of from 60° C. and 95° C.; and
c. incorporation, with stirring, and at a temperature of less than 40° C., of the biologically active agent, optionally presolubilized in an appropriate solvent.

16. A regime or regimen for the prevention and/or treatment of a dermatological disease, condition or affliction, comprising topically applying onto the affected skin area of an individual in need of such treatment, a thus effective amount of an oil-in-water emulsion as defined by claim 1.

17. The regime or regimen as defined by claim 16, comprising the treatment of psoriasis.

18. The oil-in-water emulsion as defined by claim 5, in which the propenetrating agent is selected from the group consisting of propylene glycol, dipropylene glycol and propylene glycol dipelargonate.

19. The oil-in-water emulsion as defined by claim 5, in which the propenetrating agent comprises propylene glycol.

20. The oil-in-water emulsion as defined by claim 1, in which the biologically active agent comprises clobetasol 17-propionate.
